(19) United States
(12) Patent Application Publication
(10) Pub. No.: US 2021/0031011 A1
(43) Pub. Date: Feb. 4, 2021

(54) CANNULA SYSTEM AND METHOD FOR DISCHARGING THE VOLUME OF A HEART

(71) Applicant: Berlin Heart GmbH, Berlin (DE)

(72) Inventors: Ares K. Menon, Berlin (DE); Roland Hetzer, Berlin (DE)

(73) Assignee: Berlin Heart GmbH, Berlin (DE)

(21) Appl. No.: 17/046,244

(22) PCT Filed: Apr. 9, 2019
(86) PCT No.: PCT/EP2019/058918

§ 371 Date: Oct. 8, 2020

(30) Foreign Application Priority Data
Apr. 9, 2018 (EP) 18166385.7

(51) Int. Cl. A61M 27/00; A61B 17/34

(52) U.S. Cl. CPC A61M 27/002; A61B 2017/00252; A61M 2210/125; A61B 17/3421

(57) ABSTRACT

A cannula system for puncturing the heart is provided, comprising a cannula and a trocar. The cannula comprises a cannula shaft with a heart-side inlet and a pump-side outlet. The trocar has a trocar shaft which can be inserted into the lumen of the cannula and which comprises a puncturing tip, wherein the puncturing tip can completely cover the inlet opening of the cannula.

### CROSS-REFERENCE TO RELATED APPLICATIONS

This application is a 371 nationalization of international patent application PCT/EP2019/058918 filed Apr. 9, 2019, which claims priority under 35 USC § 119 to European patent application 18166385.7 filed Apr. 9, 2018. The entire contents of each of the above-identified applications are hereby incorporated by reference.

### BRIEF DESCRIPTION OF THE DRAWINGS

There are shown in:

FIG. 1 a schematic representation of a cannula system or pump system;

FIGS. 2A and 2B an embodiment of a cannula system with a planar inlet edge;

FIGS. 3A and 4B an embodiment of a cannula system with an oblique inlet edge;

FIG. 4 an embodiment of a cannula system with a chamfered inlet edge; and

FIGS. 5A and 5B different embodiments of a cannula system with a sealing lip;

FIG. 6 an embodiment of a cannula system with an expandable cavity;

FIG. 7 a further embodiment of a cannula;

FIG. 8 a further embodiment of a cannula; and

FIGS. 9a to 9c an embodiment of an arrangement of a pressure measurement conduit.

### DETAILED DESCRIPTION

The subject-matter of the present application is a cannula for relieving the heart, a cannula system which comprises such a cannula as well as a trocar, a heart pump system which comprises a heart pump as well as at least one cannula according to the application, and further a method for the volume relief of a heart.

With regard to acute left-heart failure after a myocardial infarction, decompensation of a heart insufficiency, or with regard to other pathologies of the left-heart function with decompensation, a pumping weakness of the left ventricles arises, this amongst other things having three effects:

Firstly, a dilatation of the left ventricle with an increased volume loading and pressure loading occurs. Secondly, an undersupply of the bodily circulation with oxygen and nutrient-rich blood, an acidification of the tissue (acidosis) as well as a threat of organ failure occur. Thirdly, a blood congestion in the pulmonary circulation with an increased pulmonary capillary pressure as well as pulmonary hypertension and a threatening lung oedema occur.

Given the occurrence of the left-heart failure, often intra-aortal balloon pumps (IABP), but also extracorporeal heart assistance systems are applied for an extra-corporeal membrane oxygenation (ECMO). The access to large body vessels is herein created at least via the blood vessels of the groin. By way of this, a sufficient circulation in the patient can be ensured within a few minutes. Since, in the case of an acute left-heart failure, pulmonary congestion with an pulmonary oedema is often present, an oxygenator is connected is series for an improved supply of oxygen. However, with left heart failure, the insufficient pumping behaviour of the left ventricle is the cause and the poor oxygenation is only secondary. Despite this, ECMO has also asserted itself even with the patient cohort with primarily left-heart failure, since it recreates the marginal or absent circulation in a rapid and effective manner. For this reason, the term extra-corporal life support (ECLS) has been established, in order to distinguish these patient cohorts from the pure ECMO lung failure cohorts. With ECMO, a large body vein is selected as a feed flow to the pump or to the oxygenator and a large artery is selected as an inflow of the blood into the body circulation. A direct volume relief of the left ventricle which is severely compromised in contraction does not occur on account of this cannulation technology. Hence a possible improvement of the left-ventricular contractility is hardly possible.

Further described methods for relieving the left ventricle, e.g. via the left atrium by way of a catheter or an atrio-septectomy cannot achieve an efficient relief of the left-ventricular cavum. For this reason, the chances of a patient recovering his left-ventricular contractility solely by way of ECLS are very low.

However, one effective possibility is provided by left-ventricular assist systems, so-called left-ventricular assist devices (LVAD). These however are complex with regard to implantation technology and in many cases are not immediately implanted given an acute left heart decompensation. Furthermore, LVADs cost a multiple of the aforementioned ECMO, IABP or ECLS pump systems.

Concerning the left-ventricular assist devices, the puncturing of the heart is of great significance. Here, on implantation, usually the thorax is exposed, in order on the one hand to puncture the heart and on the other hand to attach a suture ring in the region of the puncture, for fastening the pump to the heart.

It is the object of the present invention to provide an efficient, easily implantable system for the rapidly useable and reliable implantation on the heart, so that a rapid volume relief of the left ventricle occurs and the contractility of the left ventricle can hence also be improved.

The object is achieved with the help of the cannula systems which are mentioned in the claims, as well as a heart assist system which is designed in conjunction with the cannula system. Furthermore, a method for volume relief of the left-side heart is disclosed.

The cannula system according to the application, for the puncture of the heart, comprises at least one cannula and a trocar which is designed for this cannula. The cannula comprises a cannula shaft with a heart-side inlet which comprises an inlet edge which delimits an inlet opening. A pump-side outlet is located at another end, wherein a lumen extends between the inlet and the outlet. What is meant by a pump-side outlet is herein to be understood in that the outlet of the cannula is designed in a manner such that this can be connected to known pump systems, e.g. short-term centrifugal pumps. In particular, in some embodiments, this can mean that either the cannula can be arranged on a pump in a direct manner or via a connector or that a connector which is arranged on the pump can be connected directly to the cannula.

In some embodiment examples, such types of cannulae are similar to Berlin Heart Excor cannulae, in particular the Berlin Heart Excor Apex cannulae. The cannula can thus be manufactured for example from silicone, wherein the silicone can have a wall thickness between 1 and 5 mm. An alternative material is for example polyurethane (PU). Herein, the polyurethane in some embodiments is provided with a coating, for example in order to reduce the risks of thrombosis formation.

The cannula system further comprises a trocar. This trocar comprises a trocar shaft with a puncturing tip and which can be inserted into the lumen from the outlet of the cannula shaft. The puncturing tip is herein preferably configured in a manner such that this can pierce a myocardium, i.e. that this can pierce the apex of the heart in the region of the left ventricle. In some embodiment examples, the puncturing tip is therefore formed in a cone-shaped or approximately cone-shaped manner. In this manner, the myocardium tissue is not only pierced but scarified and the puncturing tip can be pushed slowly through the myocardium into the left ventricle on account of the cone shape. Herein, the tissue of the apex is displaced in a gentle manner. Hence a myectomy does not take place, i.e. there is no resection of apex tissue, but merely a displacement of the heart tissue. This for example in the case of short-term applications has the advantage that the heart muscle can recover more quickly (even after an explantation of the cannula). This advantage comes to the forefront in particular in the case of paediatric applications, since the displacement of the heart muscle (also due to the smaller size of children's hearts) permits a more rapid recovery than after a myectomy.

According to the application, the trocar shaft is longer than the lumen of the cannula, so that the puncturing tip which protrudes at the inlet of the cannula shaft lies opposite a part of the trocar shaft which projects out of the outlet. In one puncturing configuration, the puncturing tip projects out of the inlet of the cannula. The trocar shaft further has a circumference and a geometry such that the trocar shaft or the puncturing tip completely covers the inlet opening. This essentially means that the trocar shaft or the puncturing tip completely seal off the inlet, so that given an inserted trocar in the puncturing configuration, no fluid whatsoever can enter the lumen through the inlet of the cannula shaft or given a common insertion of the cannula and the trocar which is arranged in the cannula, no apex tissue can collect in a cavity between the trocar and the cannula. Hence the danger of thrombosis at a later point in time is increased compared to other puncturing methods.

On account of the configuration of the cannula and trocar which is disclosed here, it is possible to connect the cannula to the heart without opening the thorax. For this, the trocar is firstly inserted into the cannula and the puncturing configuration is pushed. The cannula system is subsequently advanced for example below the costal arch or through the intercostal space to the apex, wherein the puncturing tip is advanced further on the apex after reaching the correct location on the heart and penetrates the left ventricle. Since the puncturing tip at the greatest circumference essentially corresponds to the circumference as the inner wall of the inlet and the wall thickness of the cannula is comparatively low compared to the diameter of the lumen of the cannula, the inlet of the cannula itself can be pushed through the myocardium into the left ventricle even in conjunction with the trocar in the apex.

Further embodiments and embodiment examples can be derived from the subsequent descriptions and figure descriptions.

Further embodiments for the method of volume relief and further embodiments of the invention are firstly described hereinafter.

Concerning the method for pressure relief according to the application, the cannula systems which are described here can be used. By way of example, a puncturing or cannulation method during the implantation, as well as an explantation method, are described hereinafter.

In order to achieve a rapid relief of the left ventricle, the tip of the left ventricle (apex) is exposed below the costal arch or through the intercostal space (4th or 5th ICR) with the help of a minimal invasive access via an antero-lateral mini-thoractomy. (This corresponds to an operative access as with a transapical TAVR). The cannula is placed by way of a Seldinger technique. First, the guide wire is applied, by way of a puncture needle, on the apex and pierces it. The wire (pigtail) is placed in the LV under radiological illumination. The trocar which is inserted into the cannula is subsequently threaded onto the guide wire and is led to the apex. Since the apex has already been punctured by the guide wire, the puncturing tip now displaces the tissue of the myocardium in the region of the apex and widens an opening until the opening has been widened to the size of the cannula. A removal of vital heart muscle tissue (myectomy) is not therefore necessary. Although myocardium tissue is also injured, the tissue, however, is displaced in an essentially gentle manner and can simplify a later recovery of the tissue. On account of the widening of the tissue, the cannula now can be advanced into the ventricle up to the fixation edge (suture ring) which is then connected to the heart by way of individual button sutures. After the sutures which were priorly placed on the ventricle have been fixed to the cannula and this connected to the heart itself in a blood-tight manner, the trocar can be retracted and hence the cannula can be bled of air, in order to subsequently be temporarily clamped. Optionally, further continuing sutures can be attached to the suture ring for an improved sealing with regard to blood.

In further embodiment examples, spacers or one or more spacers can be incorporated into the rib intermediate space or intercostal space on both sides of the cannula, in order to prevent a possible sharp bending or a compression of the cannula due to the ribs. Alternatively, the cannula can be provided with wall thickenings which prevent a compression due to the ribs. The cannula is subsequently deaerated and connected for example to a commercially available centrifugal short-term blood pump, such as e.g. a Rota-flow of Marquet, a Hemopump of Sorin, a Deltasteam DP3 of Medos/Xenios. However, the system can also be connected for example to a para-corporal displacement pump such as Berlin Heart Excor.

The pump which is selected for the pump system is further connected to a further cannula, the so-called arterial outflow cannula. This arterial cannula can either be connected percutaneously or surgically to the large groin artery or surgically to the subclavian artery. Furthermore, in some embodiment examples concerning a patient, inasmuch as he is already connected to an ECMO/ECLS due to his poor circulation situation, the arterial access of the ECMO/ECLS can firstly be used as an outflow location, and the venous access is removed with the cannula system after a successful establishment and cannulation of the apex. Blood can therefore be led out of the left ventricle via the established system and hence a direct relief of the left ventricle (apex cannula) and an assistance of the circulation system (arterial outflow cannula) can be achieved. Hence the cannula together with the pump and the outflow cannula represent a short-term VAD system with a possible assist time of up to 30 days (depending on the applied pump).

In order to be able to optimally control the therapy, an additional pressure measurement conduit can be integrated into the cannula. This pressure measurement conduit runs out in the ventricle at the tip of the cannula. This pressure measurement conduit is then connected at the outlet side to a commercially available pressure recording system and can thus be connected to a common monitor for haemodynamic monitoring. This pressure measurement can continuously display the measurement of the LVEDP (left ventricular end diastolic pressure). This for example can help in the control of the optimal relief of the left ventricle e.g. adaptation (increase or lowering of the delivered quantity of blood volume per minute). In particular, this plays a role in weaning the patient off the system after the heart has recovered from the heart weakness.

With a recovery of the heart function, it is necessary to reduce the blood flow through the cannula or pump in steps. On reducing the flow, more blood volume is left back in the left ventricle, in order to see whether the heart comes to terms with the increased loading without the filling pressure of the left heart ventricle (LVEDP=left ventricular end diastolic pressure) increasing. In order to be able to estimate the contraction performance of the heart under increased loading, it is therefore preferably the LVEDP which is used. If the heart function has recovered, then the apex cannula and herewith the VAD system can again be removed via the minimally invasive access.

For this, the apex region is again rendered accessible. The cannula is clamped, in order to have no more flow of blood via the system. Since a myectomy is not effected, a purse-string suture can be placed via felts which have already been priorly placed on implantation, said purse-string suture then closing the entry location of the cannula on the apex during and after withdrawing the cannula, by way of the surrounding heart muscle tissue being contracted. The cannulation location is additionally secured by felt-reinforced sutures.

In an embodiment, the trocar comprises a peripheral sealing lip which is preferably arranged at the transition between the puncturing tip and the actual trocar shaft. This sealing lip is arranged in a manner such that it at least partly covers the inlet edge, preferably completely encompasses and covers the inlet edge. A sealing lip which is arranged between the puncturing tip and the trocar shaft amongst other things has the advantage that a tactile feedback is produced on leading the sealing lip out of the cannula shaft, and the trocar can be pushed through the lumen much more simply after the sealing lip has left at the inlet of the cannula. With the subsequently effected retraction of the trocar shaft, the sealing lip completely seals of the inlet opening, so that the inlet opening is completely covered, even if the trocar shaft has a circumference which is smaller than the circumference of the inner side of the lumen. The sealing lip can herein be designed differently in various embodiments.

The sealing lip can therefore be arranged for example in a collar-like or flange-like manner on the transition between the puncturing tip and the trocar shaft. The sealing lip however can also form an extension of the puncturing tip, so that the sealing lips extend an essentially cone-shaped fashion of the puncturing tip, i.e. project slightly in the direction of the outlet and to the outside.

In a further embodiment, the inlet edge is chamfered and comprises a chamfer in the axial direction towards the inlet opening. This embodiment is particularly advantageous if the puncturing tip (or also the puncture tip) terminates with the inlet edge in a flush manner. For this, in some embodiments the puncturing tip can likewise be chamfered and a chamfer angle of the puncturing tip and a chamfer angle of the chamfer of the inlet edge can differ from one another by less than 30°, in particular by less than 20° or 10° and in particular are essentially equal. In other words, given a similar chamfer angle, the inlet edge is to be seen as an extension of the puncturing tip of the trocar. By way of this, an insertion of the cannula into the apex in a manner which is particularly gentle on the tissue is rendered possible. For example, angles of larger than 30°, measured in the plane transverse to the longitudinal axis of the cannula, preferably angles of more than 45°, particularly preferably greater than 60° lend themselves as chamfer angles. A gentle insertion of the cannula system into the left ventricle is ensured in this manner.

In a further embodiment, the puncturing tip of the trocar comprises an expandable cavity, and the puncturing tip covers the inlet opening when the cavity is in the expanded state. In some embodiments one envisages the puncturing tip at least partly, preferably completely covering the inlet edge in the expanded state of the cavity. As explained in the other embodiment examples, a good sealing is achieved between the trocar and the cannula and at the same time an improved insertion of the cannula system into the left ventricle is ensured.

In order to expand the cavity, one can envisage a media inlet being present at the outlet-side end of the trocar, via which inlet a medium can be filled into the cavity and expands this. For example saline solutions can be considered as media or also air in other embodiment examples.

In many cases, the trocar or the puncturing tip of the trocar comprises a hard material, such as for example steel, or a hard bio-compatible plastic. However, it is also envisaged for the trocar shaft to have a sufficient flexibility, in order to be led through the intercostal space onto the left apex. The trocar can comprise for example a jacket of a flexible bio-compatible plastic.

In some embodiments, one envisages a suture ring for the connection of the cannula to the heart being arranged on the outer side of the cannula shaft in the proximity of the inlet. Herein, the suture ring is connected to the cannula shaft for example with a material fit. The suture ring can consist for example of a felt or comprise this, so that the suturing is possible in a simple way and manner.

In a further embodiment, a trocar marking is provided on the trocar shaft, said marking being able to be arranged in a manner corresponding to a cannula marking which is arranged on the cannula. This means that the trocar marking can be arranged for example congruently with the cannula marking or at the same height (i.e. transversely to the longitudinal axis of the lumen). These markings can be arranged in a manner such that by way of bringing the markings to coincide, it can be recognised when the puncturing tip of the trocar covers the inlet opening. In particular, in embodiments in which a sealing lip or a chamfering of the inlet edge which corresponds to the chamfer angle of the puncturing tip is provided, this can distinguish that location, in which the puncturing tip terminates with the inlet edge in a flush manner, or in which the sealing lip bears on the inlet edge and seals the lumen of the cannula.

In a further embodiment, a further trocar marking is provided, this with regard to the longitudinal axis of the trocar shaft and compared to the first trocar marking being arranged further to the outlet side. By way of this, it can be recognised that when the further trocar marking has been brought to coincide with the cannula marking, it can be recognised that the trocar must now be pulled back in the direction of the outlet, in order to accomplish a good sealing of the inlet opening by way of the puncturing tip or the trocar shaft.

In some embodiment examples, one can envisage the trocar marking of the trocar being a stop which projects out of the trocar shaft, and the cannula marking either being an outlet edge of the cannula shaft or providing an abutment for the stop in the region of the outlet.

In a further embodiment, one envisages a metal sleeve or a composite material sleeve being incorporated into the cannula shaft in the region of the inlet edge of the cannula. In some embodiments, the metal sleeve or the composite material sleeve can be incorporated into the cannula shaft such that this projects out of the silicone or plastic jacket of the cannula and the uncovered metal or composite material sleeve forms the inlet edge. In other embodiments, one can envisage the metal sleeve or the composite material sleeve being incorporated completely into the cannula shaft and being covered at least by a thin material layer of the cannula material. In this embodiment, on the one hand a good biocompatibility is ensured on account of the material of the cannula shaft and on the other hand a corresponding stiffening of the inlet edge is ensured, which can be advantageous on inserting the cannula system into the left ventricle.

As already mentioned, the cannula system alternatively or additionally to the aforedescribed features comprises a pressure measurement conduit which is led on the cannula shaft, in the lumen of the cannula shaft or within the wall of the cannula shaft. Examples as to how a pressure measurement conduit is arranged on the cannula can be derived from the International patent application PCT/EP2017/076811 which to the full extent is adopted as a constituent of this application. Herein, the cannula is a pressure measurement conduit with a pressure inlet and a pressure outlet, wherein the pressure inlet of the pressure measurement conduit is arranged at the heart side of the suture ring. This means that the pressure inlet is arranged essentially between the suture ring and the (cannula) inlet. Herein, the cross section of the pressure measurement conduit is smaller than the cross section of the cannula lumen. The cross section of the pressure measurement conduit is preferably at least five times or ten times smaller than the cross section of the lumen of the cannula. In this manner, it is ensured that the pressure measurement conduit only slightly increases the diameter of the cannula. By way of the pressure measurement conduit, it is possible to permanently monitor the pressure within the left atrium, without having to insert a separate pressure measurement conduit temporarily into the cannula, for example via a port. The pressure measurement conduit is preferably led along an outer side of the cannula shaft. Herein, the pressure measurement conduit can be welded or bonded for example to the outer side of the cannula shaft. In a further special embodiment, the pressure measurement conduit is led in the lumen of the cannula. In a further special embodiment, the pressure measurement conduit is arranged in the cannula shaft wall as a separate lumen. The pressure measurement conduit is configured such that it is connectable to an external pressure measuring system, for example a commercially available blood pressure measuring system such as the intensive monitors of Philips, HP or Siemens. The pressure outlet is provided for example with an adapter which is standardised and can be coupled to the selected system. Although in numerous embodiments the pressure measurement conduit is materially connected to the outer side of the cannula shaft, the pressure measurement conduit can project beyond the outlet of the cannula at the outlet side and be significantly longer than the cannula shaft, so that a coupling to an external pressure measurement system is possible.

Alternatively to, or in combination with the pressure measurement conduit, a pressure sensor, such as for example a mechanical-electromagnetic sensor can be arranged in the proximity of the inlet of the cannula. A pressure sensor which is provided with a membrane can also be arranged on the inlet of the cannula.

If, after the implantation of the cannula and pump system which are presented here, an oxygen problem continues to be present, then an oxygenator can be connected serially into the outflow cannula. Further embodiments and supplements to the cannula system presented here result from the subsequent embodiment examples and the claims.

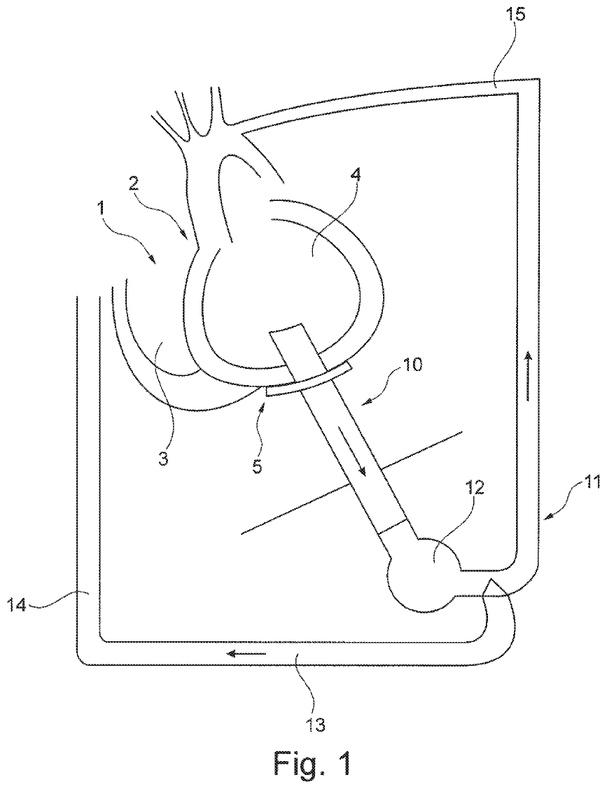

By way of FIG. 1, it is schematically described as to the scope in which a cannula system or a pump system according to this application can be applied.

A circulation system 1 with a heart 2 is shown in FIG. 1, said heart amongst other things having a right ventricle 3 and a left ventricle 4. An opening through which a cannula 10 is arranged as part of a pump system 11 is located at the apex 5 of the left ventricle 4. The cannula 10 takes blood out of the left ventricle and leads this from the heart-side inlet to the pump-side outlet into the pump 12 which is coupled to a further cannula whose inlet receives the blood which is pumped by the pump and releases the blood back into the circulation for example in the femoral artery 14 or the subclavian artery 15. The outlet cannula 13 for example can be an Excor cannula or Excor vessel cannula. Furthermore, should an external oxygenation of the blood be desired, then an oxygenator can be connected between the pump 12 and the outlet cannula 13. The cannula 10 is described in more detail hereinafter by way of the subsequent embodiment examples. Depending on the field of application, the length of the cannulae 10 and 11 is between 20 and 80 cm. Lengths between 30 and 50 cm are preferably considered. The cannula enters through the skin between the inlet and the outlet and is connected to the pump 12 in an extracorporeal manner. With regard to the pump 12, it can be a LVAD or a commercially available short-term pump.

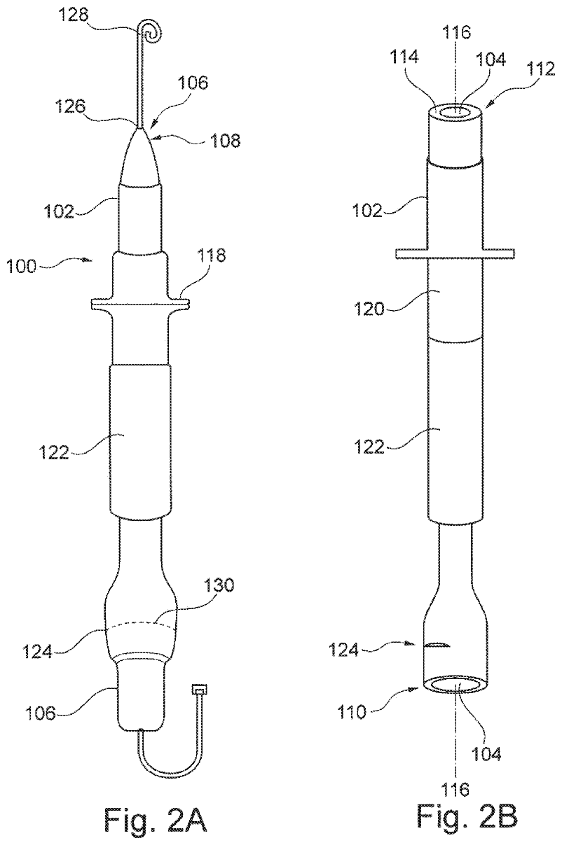

FIGS. 2A and B show an embodiment of a cannula system 100. The cannula system comprises a cannula 102 and a trocar 106 which is with a puncture tip 108 and which is inserted through the lumen 104. The lumen herein extends between the pump-side outlet 110 and the heart-side inlet 112. The inlet 112 is delimited by an inlet edge 114, wherein the inlet edge in the present embodiment example of FIG. 2 is designed in a planar manner. Planar means that the inlet edge is aligned essentially in a plane perpendicular to the longitudinal axis 116 of the cannula 102. The cannula has a length of for example 50 to 70 cm between the inlet 112 and the outlet 110. The thickness or wall thickness of the cannula in the region of the inlet 112 is for example 2 mm. The wall thickness can be less in the region of the outlet 110, in order to simplify a connection of the outlet 110 to a pump. In FIG. 2, it is schematically shown that the outlet 110 is slightly widened compared to the inlet, in order to hence be able to receive the inlet of the pump more easily. Alternatively, an additional adapter can be arranged in the region of the outlet 110, which simplifies the connection to the pump. A suture ring 118 is arranged on the outer side of the cannula 102 and can be connected to a suture ring which is not represented and which can be connected to the apex of the heart or is already connected to the heart on inserting the cannula. For this, the suture ring is designed for example in a flange-like manner and can consist for example of a felt, a fabric or of the material of the cannula shaft 120 itself or comprise these materials. The length of the cannula between the inlet 112 and the suture ring 118 can be for example 2 to 5 cm. This length is adequate in order to arrange the suture ring essentially on the outer side of the heart and to anchor the inlet securely in the inside of the ventricle of the patient. Furthermore, a reinforcement 122 which is arranged in the region of the passage of the cannula through the skin and is to encourage the ingrowth of the cannula in the region of the skin is located between the suture ring 118 and the outlet 110. By way of this, the risk of infection to the patient is reduced. Although the cannula in FIG. 2 is represented without colour, the cannula can be manufactured for example from a transparent material. For example, the cannula can essentially comprise a silicone shaft. Here, silicone is transparent. A marking 124, whose function with be dealt with in more detail is arranged in the region of the outlet.

The trocar 106, as already mentioned, comprises a puncturing tip 108. The puncturing tip extends between the inlet edge 114 and the tip 126 over a length for example of 3 cm. Herein, the puncturing tip of the trocar tapers conically or in a cone-shaped manner from the inlet 112 to the tip 126. The puncturing tip at the inlet-side end has a diameter which is identical or slightly larger than the diameter of the cannula in the region of the inlet 112 without the inserted trocar. In the present example, the trocar can consist for example of a biocompatible plastic and herein the tip can be shaped in a manner such that this is capable of displacing the tissue of the myocardium in the region of the apex. A guide wire 128 is further to be recognised in the present example. The cannula can be applied for example by way of the Seldinger technique. The guide wire is firstly applied on the apex and pierces this. The trocar 106 which is inserted into the cannula 102 is subsequently threaded onto the guide wire and led to the apex. Since the apex has already been punctured by the guide wire, the puncturing tip now displaces the tissue of the myocardium in the region of the apex and widens an opening until the opening has been widened to the size of the cannula. Although herein myocardium tissue is also injured, the tissue however to an essential extent is displaced and can simplify a later recovery of the tissue. By way of the widening of the tissue, the cannula now can be advanced into the ventricle and the suture ring 118 can be sutured to a suture ring which is arranged on the heart. After the fastening of the cannula to the heart has been effected, the guide wire 128 and the trocar 106 can be pulled out of the cannula and these can be temporarily clamped until connection to a pump. In the inserted state the trocar, a marking 130 of the trocar lies at the same height as the marking 124 of the cannula 102. By way of this, the cardiologist can examine externally as to whether the trocar has been inserted so far into the cannula that the puncturing tip projects completely out of the inlet, and the trocar shaft or the outlet-side end of the puncturing tip terminates with the inlet or the inlet edge. Since the puncturing tip 108 now terminates with the inlet 112 in a flush manner, the insertion of the cannula into the ventricle is significantly simplified compared to existing methods.

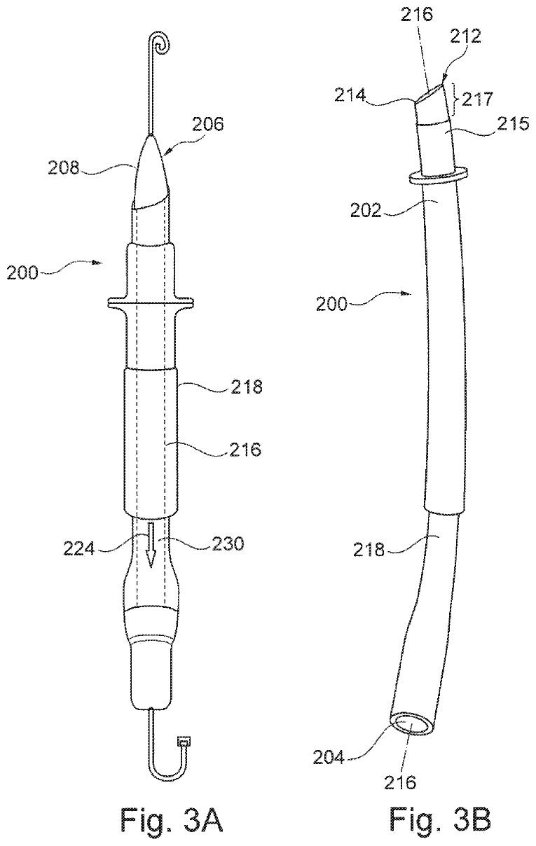

A further cannula system 200 is shown in FIG. 3. The cannula system 200, similarly to the cannula system 100, comprises a cannula 202 with a lumen 204, around which a trocar 206 has been introduced. In the inserted state, a puncturing tip 208 of the trocar 206 projects out of the inlet 212 of the cannula 202 and completely covers this. Differently to the embodiment example of FIG. 2, the inlet edge 214 in the present example is not designed in a planar manner, but in an oblique manner, i.e. the inlet edge is formed in a plane at an angle of less that 90° with respect to the longitudinal axis 216 of the cannula. On account of the oblique embodiment of the inlet edge, an improved flow of blood can be effected in some embodiments. As can be recognised in FIG. 3B, the upper inlet section 217 which is formed between the inlet edges 214 in a connection suture 215 is manufactured from a different material than the cannula shaft 218. In the present example, the upper inlet section is formed from titanium or a titanium sleeve which is incorporated into the cannula shaft. The incorporation can be effected by way of moulding or a material-fit connecting. The metal sleeve, for instance of titanium, for example can have improved biocompatible characteristics. In FIG. 3, an alternative form of a marking 224 is also recognisable, and this marking has been brought to coincide with the marking 230 of the trocar which lies therebelow. The marking is not a marking which is designed purely transversely to the longitudinal axis 216, but a two-dimensional pictogram. Due to the fact that the trocar comprises a pictogram which corresponds to the pictogram of the cannula, by way of bringing the marking to coincide, one succeeds in the puncturing tip projecting through the inlet at a certain orientation and hence a covering of the inlet can be achieved given an oblique embodiment of the inlet edge 214.

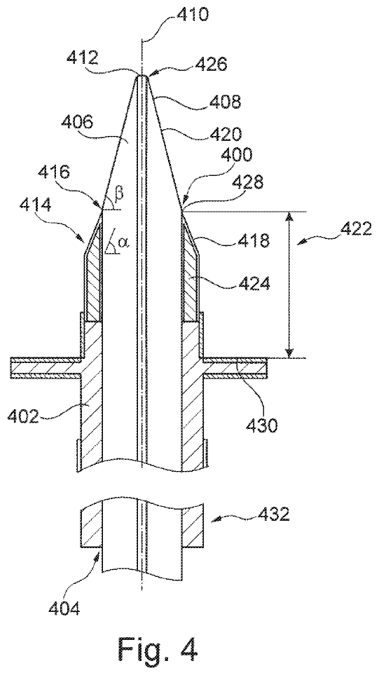

FIG. 4 also shows a cannula system. The cannula system 400 apart from the cannula 402 comprises a trocar 406 which is with a puncturing tip 408 and which is pushed through the lumen 404 of the cannula 402. The trocar further comprises an opening 412 along the longitudinal axis 410 of the cannula (as well as of the trocar), through which opening a guide wire can be pushed, in order to implant the cannula system with the help of the Seldinger technique. The inlet 414 of the cannula 402 comprises an inlet edge 416 which runs essentially transversely to the longitudinal axis 410, but in contrast to the embodiment example of FIG. 2 comprises a chamfer 418. Herein, the chamber extends over a length of approximately 4 mm. Herein, the chamfer angle α, considered transverse to the longitudinal axis 410, is 75°. Due to this tapering of the inlet edge in the manner of a truncated cone, and a correspondingly designed puncturing tip 408 which tapers at the same chamfer angle β=75°, the inlet edge 416 is now designed as a lengthening of the puncturing tip 408, inasmuch as the trocar is placed correctly within the cannula. The respective placement can be effected for example via the markings which are mentioned by way of example in FIG. 2 or 3. Due to the fact that the surface 420 of the puncturing tip overlaps with the chamfer 418 in an essentially continuous manner, and the trocar or the puncturing tip completely covers the inlet opening, a particularly gentle insertion of the cannula into the apex or into the ventricle is rendered possible. In order to be able to manufacture the geometry of the chamfer in a precise manner, the upper inlet section 422 can comprise for example a titanium sleeve 424 which has been chamfered for example by way of milling or etching. Herein, the silicone material of the remaining cannula 402 can be completely peripherally moulded around the titanium sleeve 424 or this sleeve can extend out of the upper part of the silicone cannula. Although in the present example the chamfer angles α and β are essentially identical, they can also be selected differently from one another. Other geometries, such as for example an angle 11 which continuously increases from the upper end of the tip 426 of the puncturing tip 408 to the lower end 428 of the puncturing tip can also be selected, so that the puncturing tip with regard to pointedness is essentially more pointed than in the region of the inlet edge. The inlet edge 416 is herein essentially distanced 4 cm from a suture ring 430 which is formed from textile. The length of the cannula 402 (and accordingly the length of the trocar) between the inlet 416 and the outlet 432 is for example 65 cm. The trocar can be accordingly be 10 to 20 cm longer.

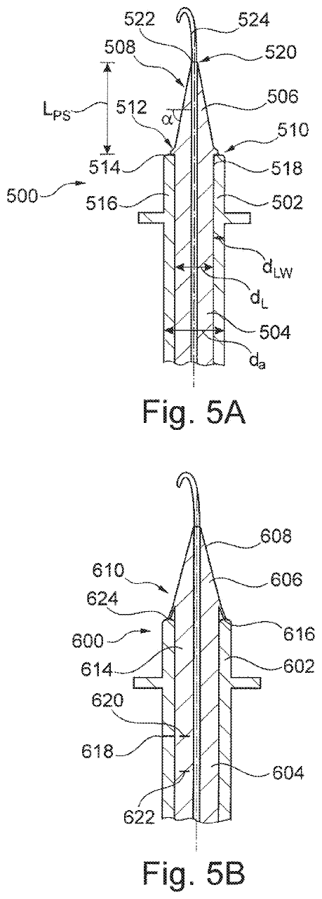

FIG. 5 each show a cannula system, concerning which the trocar comprises a sealing lip at the lower end of the puncturing tip.

The cannula system 500 comprises a cannula 502, in whose lumen 504 a trocar 506 with a puncturing tip 508 is arranged. A sealing lip 512 which peripherally covers the inlet edge 514 of the inlet 516 is located at the lower edge 510 of the puncturing tip. The lumen 506 has a diameter dₗ, the wall thickness d_{lw} is 3 mm. The sealing lip 512 is arranged at the lower end 510 of the puncturing tip in a manner such that this extends out of the puncturing tip in the manner of a flange and projects beyond the inner edge 518 of the inlet edge 514 by approximately 1.5 mm. By way of this, the inlet 516 is closed over the complete periphery and foreign bodies cannot penetrate into the lumen 506. The puncturing tip 508 has a length L_{PS} of 30 mm. The puncturing tip 508 herein tapers at an angle α=80° from the lower end 510 of the puncturing tip to the upper end 520. A lumen 522 through which a guide wire 524 can be threaded is located in the inside of the trocar. The bead-like edge in the form of a sealing lip 512 consists of a comparatively soft material, so that the trocar can be pulled completely through the lumen 506 in the direction of the outlet, and conversely can also be inserted into the lumen 506 from the outlet which is not represented in FIG. 5A, and can be led out to such an extent that the sealing lip 512 bears on the inlet edge 514 over the whole periphery and covers this. Since the sealing lip covers a somewhat smaller area than the surface which is defined by the outer diameter of the inlet edge 514, the outer diameter da of the cannula is slightly larger than the diameter of the puncturing tip 508 in the region of the sealing lip 512. However, on account of the angle α of the puncturing tip, the myocardium tissue is widened far enough so that the cannula including the sealing lip can be inserted. In the present embodiment example, the cannula shaft is manufactured for example of silicone. The trocar can be manufactured for example of a biocompatible polyurethane, wherein this in the region of the sealing lip is capable of widening the inner wall of the silicone cannula, i.e. the trocar 506 does not change its shape, whereas the inner wall of the silicone cannula 502 is displaced outwards on leading the trocar through the lumen 506. In order to ensure a corresponding correct arrangement of the trocar to the inlet edge 514, one can fall back for example on the marking. Apart from the markings which have already been mentioned with regard to FIGS. 2 and 3, a further trocar marking can additionally be present, this marking signalising that the trocar has been pushed too far out of the inlet and must now be retracted. This for example can be a trocar marking which is arranged proximally of the marking of FIG. 2 or 3.

An alternative cannula system 600 is shown in FIG. 5B. This, apart from the cannula 602 with the cannula lumen 604, comprises a trocar 606 with a puncturing tip 608. This embodiment example also comprises a sealing lip 610. The sealing lip is herein selected such that this adapts essentially to the shape of the puncturing tip 608 and lengthens or extends this in the lower region 612. The sealing lip 610 is hence designed in the manner of a collar. If the trocar 606 is now pushed from the outlet which is not shown in FIG. 5B, through the cannula lumen 604 in the direction of the inlet, then the collar 610 is applied onto the outer sides of the trocar shaft 614 situated below the puncturing tip 608, and can be simply pushed through the lumen. The collar must firstly be led out of the lumen 604 over the complete periphery and the trocar subsequently retracted slightly in the direction of the outlet, in order to now unfold the now unfolded collar (as is represented in FIG. 5B), and to now bring it to coincide with the inlet edge 616. A sealing of the inlet edge by way of the sealing lip 610 is therefore ensured. For this purpose, the cannula comprises a marking 618 and the trocar a first marking 620 and a second marking 622. The trocar is firstly pushed through the lumen until the marking 622 coincides with the marking 618. In this manner, it can be clearly recognised by the operator that the collar 610 has been completely led out of the lumen and could hence unfold. The trocar is subsequently retracted until the marking 620 coincides with the marking 618, so that it can be recognised by the operator that the lower edge of 624 of the sealing lip 610 is in contact with the inlet edge 616 and hence reliably covers this so that no tissue can collect in the lumen 604 of the cannula 602.

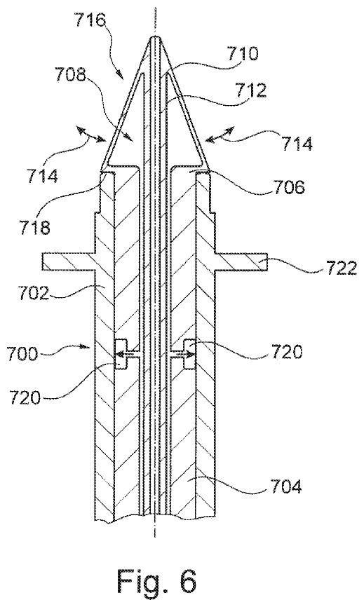

A further embodiment example of the cannula system 700 is represented in FIG. 6. The cannula 702 comprises a lumen 704, through which the trocar 706 which is represented in FIG. 6 can be inserted. The trocar 706 comprises an expandable cavity 708 which is delimited by an outer wall 710 and an inner wall 712. A cavity inlet 714 through which a medium, such as for example a saline solution or air can be filled into the expandable cavity 708, is represented in a schematic manner. As is indicated by way of the arrows 714, a puncturing tip 716 is formed on expanding the cavity 708, concerning which tip the outer diameter increases in the region of the inlet edge 718 and hence covers this. A secure covering of the lumen 704 can also be effected by way of this, and no tissue can collect between the puncturing tip and the cannula shaft 702. The cavity can comprise further side chambers 720 which press the trocar onto the inner wall of the cannula shaft, in order to additionally effect a non-positive fit of the trocar within the cannula.

After the trocar has been used for puncturing the apex, the cannula inserted into the ventricle and the suture ring 722 connected to the respective suture ring on the heart, the medium can be discharged via the cavity inlet 714, the trocar or the cavity contracts and the trocar can be withdrawn through the lumen 704 in a simple way and manner. The cannula is subsequently temporarily clamped.

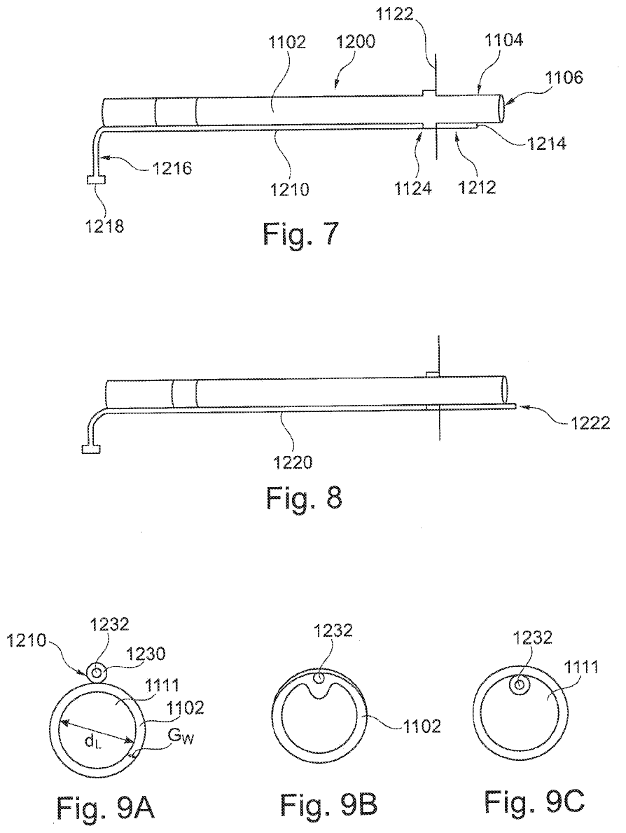

A further variant of a cannula is described by way of FIG. 7. The cannula 1200 which is represented in FIG. 7, amongst other things comprises a pressure measurement conduit 1210 which extends along the cannula shaft 1102. The inlet configuration of the cannula, as is represented by way of example in the previous embodiment examples, is not explicitly represented in the subsequent figures, so that it is the pressure measurement conduit which is dealt with here. The embodiment examples of FIGS. 1 to 6 can therefore be combined with the embodiment examples of FIGS. 7 to 9.

The pressure measurement conduit 1210 comprises a pressure measurement conduit inlet 1212 with a distal opening 1214 which lies proximally of the distal opening 1106, but distally of the distal end of a drainage basket opening 1110. The drainage basket is optional. The pressure measurement conduit outlet 1216 lies proximally of the proximal opening 1114 of the cannula and comprises an adapter 1218 which is designed for the connection to an external pressure measuring system. For example, the connection can be a snap fit connector or a Luer lock for the connection to an external pressure measuring system. Such adapters have been known for some time in the state of the art. One succeeds in the pressure in the ventricle being able to be measured at every point in time by way of the pressure measurement conduit 1210 which is integrated into the cannula 1200. The threading of an additional pressure measurement conduit into the cannula itself is done away with by way of this, such having to be carried out at regular or irregular intervals in order to determine the pressure conditions in the ventricle. In the present example of FIG. 7, the pressure measurement conduit 1210 runs outside the cannula shaft 1102 and is fastened to this with a material fit. The pressure measurement conduit 1210 can be either bonded or welded to an outer wall of the cannula shaft 1102. The pressure measurement conduit 1210 is herein led through an opening 1124 or a cut-out of the suture ring 1122, so that it is ensured that the pressure measurement conduit inlet 1212 comes to lie within the ventricle. Although in the present embodiment example the distal opening 1214 of the pressure measurement conduit inlet 1212 lies proximally of the distal opening 1106, as is shown in FIG. 8, in another embodiment example a distal opening 1224 of a pressure measurement conduit inlet 1222 can lie distally of the distal opening 1106 of the cannula. In other embodiment examples, the distal opening of the pressure measurement conduit inlet can terminate with the distal opening 1106 of the cannula in a flush manner.

The different positionings of the pressure measurement conduit in cross section are to be represented by way of FIGS. 9A to 9C. In FIG. 9A, the wall of the cannula shaft 1102 is represented with the wall thickness G_{w}. The wall thickness preferably has a constant thickness between 1 to 5 mm. The lumen 1111 of the cannula 1100 has a diameter D_{L} of 0.3 to 2 cm. The pressure measurement conduit 1210 is arranged outside the lumen 1111 and comprises a separate conduit wall 1230. The conduit wall can herein preferably have a constant wall thickness between 0.5 to 3 mm. The fluid, which is blood in the present example, can penetrate the distal opening into the lumen 1232, get to the adapter and thus be led to a pressure measurement by way of a pressure meter in an external pressure measuring system. Alternatively, a pressure measuring sensor can be arranged within the pressure measurement conduit. This may be a membrane which is arranged in the pressure measurement conduit 1210, wherein the force which acts upon the membrane can be converted into a pressure. For this, the deflection or the force which acts upon the membrane can be led further to an external evaluation system. The further leading can be carried out for example by way of an electrical lead which runs within the wall 1230. However, in order to ensure a high flexibility, the actual pressure measurement often takes place in an external pressure measuring system.

In FIG. 9B, the lumen 1232 is integrated into the wall of the cannula shaft 1102. In FIG. 9C, the pressure measurement conduit (similarly to in FIG. 9A) is arranged separately, which is to say not within the wall of the cannula shaft 1102, but runs within the lumen 1111. Here too, the pressure measurement conduit can be materially connected to the wall of the cannula shaft 1102.

The present document can further comprise the subject-matters of the following aspects.
1. A cannula system for the puncture or volume relief of the heart, comprising a cannula and a trocar,
   wherein the cannula comprises a cannula shaft with a heart-side inlet which comprises an inlet edge which delimits the inlet opening, and with an pump-side outlet and a lumen which extends between the inlet and the outlet; and
   wherein the trocar comprises a trocar shaft which is with a puncturing tip and is insertable into the lumen from the outlet, and the trocar shaft is longer than the lumen, so that in a puncturing configuration the puncturing tip projects out of the inlet of the cannula; and
the trocar shaft or the puncturing tip completely covers the inlet opening.
2. A cannula system according to one of the preceding aspects, wherein the puncturing tip comprises a peripheral sealing lip which at least partly covers the inlet edge.
3. A cannula system according to one of the preceding aspects, wherein the inlet edge in the axial direction to the inlet opening comprises a chamfer and the puncturing tip terminates with the inlet edge in a flush manner.
4. A cannula system according to aspect 3, wherein the puncturing tip is chamfered and a chamfer angle of the puncturing tip and a chamfer angle of the chamfer of the inlet edge differ from one another by less than 30°, in particular from one another by less than 20° or 10° and in particular are essentially equal.
5. A cannula system according to one of the preceding aspects, wherein at least the puncturing tip of the trocar comprises an expandable cavity, and the puncturing tip completely covers the inlet opening when the cavity is in an expanded state.
6. A cannula system according to aspect 5, wherein the puncturing tip at least partly covers the inlet edge when the cavity is in the expanded state.
7. A cannula system according to one of the aspects 5 or 6, wherein the trocar shaft comprises a cavity inlet which is connected to the expandable cavity, so that the cavity can be expanded via the inlet.
8. A cannula system according to one of the preceding aspects, wherein a suture ring for connecting the cannula to the heart is arranged on an outer side of the cannula shaft and the outlet is configured in a manner such that the outlet is connectable to a pump.
9. A cannula system according to one of the preceding aspects, wherein the trocar on its trocar shaft comprises a trocar marking and the cannula comprises a cannula marking, and the trocar marking and the cannula marking can be arranged to one another in such a manner that it can be recognised when the puncturing tip of the trocar covers the inlet opening.
10. A cannula system according to aspect 9, wherein a further trocar marking is present, this being able to be arranged with respect to the cannula marking such that it is recognisable that the trocar must be pulled in the direction of the outlet up to the trocar marking.
11. A cannula system according to one of the aspects 8 to 10, wherein the trocar marking of the trocar is a stop and the cannula marking is an outlet edge of the cannula shaft.
12. A cannula system according to one of the preceding aspects, wherein a metal sleeve or a composite material sleeve is incorporated into the cannula shaft in the region of the inlet edge of the cannula.
13. A cannula system according to one of the preceding aspects, wherein the puncturing tip is designed in a manner such that the myocardium can be pierced.
14. A cannula system according to one of the preceding aspects, wherein the inlet edge runs obliquely with respect to a plane which lies transversely to the longitudinal axis of the lumen of the cannula shaft.
15. A cannula system according to one of the aspects 8 or an aspect which refers to aspect 8, wherein the cannula comprises a pressure measurement conduit with an inlet and an outlet, wherein the inlet of the pressure measurement conduit is arranged at the heart side of the suture ring.
16. A cannula system according to aspect 15, wherein the pressure measurement conduit is led along the outer side of the cannula shaft or is led in the lumen of the cannula or is led in a cannula wall.
17. A pump system which comprises a heart pump, an inlet cannula and an outlet cannula or outflow cannula, wherein the inlet cannula is part of a cannula system according to one of the preceding aspects.
18. A method for the cannulation of the left-side heart, comprising the following steps:
   puncturing the apex of the heart with a guide wire;
   threading a cannula system comprising a cannula and a trocar, onto the guide wire;
   introducing the cannula system up to the apex and puncturing the apex by way of a puncturing tip of the trocar;
   displacing the myocardium by way of the puncturing tip of the trocar and inserting the inlet of the cannula into the ventricle;
   and preferably suturing a suture ring of the cannula to the outer wall of the heart.

1. A cannula system for the puncture or volume relief of the heart, comprising a cannula and a trocar,
wherein the cannula comprises a cannula shaft with a heart-side inlet which comprises an inlet edge which delimits the inlet opening, and with a pump-side outlet and a lumen which extends between the inlet and the outlet, wherein a suture ring for connecting the cannula to the heart is arranged on an outer side of the cannula shaft and the outlet is connectable to a pump; and
wherein the trocar comprises an opening for a guide wire and a trocar shaft which has a puncturing tip and is insertable into the lumen from the outlet, and the trocar shaft is longer than the lumen, so that in a puncturing configuration the puncturing tip projects out of the inlet of the cannula; and
the trocar shaft or the puncturing tip completely covers the inlet opening.

2. The cannula system of claim 1, wherein the puncturing tip comprises a peripheral sealing lip which at least partly covers the inlet edge.

3. The cannula system of claim 1, wherein the inlet edge in the axial direction to the inlet opening comprises a chamfer and the puncturing tip terminates with the inlet edge in a flush manner.

4. The cannula system of claim 3, wherein the puncturing tip is chamfered and a chamfer angle of the puncturing tip and a chamfer angle of the chamfer of the inlet edge differ from one another by less than 30°.

5. The cannula system of claim 1, wherein at least the puncturing tip of the trocar comprises an expandable cavity and the puncturing tip completely covers the inlet opening when the cavity is in an expanded state.

6. The cannula system of claim 5, wherein the puncturing tip at least partly covers the inlet edge when the cavity is in the expanded state.

7. The cannula system of claim 5, wherein the trocar shaft comprises a cavity inlet which is connected to the expandable cavity, so that the cavity can be expanded via the inlet.

8. The cannula system of claim 1, wherein the trocar on its trocar shaft comprises a trocar marking and the cannula comprises a cannula marking, and the trocar marking and the cannula marking can be arranged to one another in such a manner that it can be recognised when the puncturing tip of the trocar covers the inlet opening.

9. The cannula system of claim 8, wherein a further trocar marking is present, this being able to be arranged with respect to the cannula marking in a manner such that it is recognisable that the trocar must be pulled in the direction of the outlet up to the trocar marking.

10. The cannula system of claim 8, wherein the trocar marking of the trocar is a stop and the cannula marking is an outlet edge of the cannula shaft.

11. The cannula system of claim 1, wherein a metal sleeve or a composite material sleeve is incorporated into the cannula shaft in the region of the inlet edge of the cannula.

12. The cannula system of claim 1, wherein the puncturing tip is configured to pierce the myocardium.

13. The cannula system of claim 1, wherein the inlet edge runs obliquely with respect to a plane which lies transversely to the longitudinal axis of the lumen of the cannula shaft.

14. The cannula system of claim 1, wherein the cannula comprises a pressure measurement conduit with an inlet and an outlet, wherein the inlet of the pressure measurement conduit is arranged at the heart side of the suture ring.

15. The cannula system of claim 14, wherein the pressure measurement conduit is led along the outer side of the cannula shaft, is led in the lumen of the cannula, or is led in a cannula wall.

16. A pump system which comprises a heart pump, an inlet cannula and an outlet cannula or outflow cannula, wherein the inlet cannula is part of a cannula system comprising a cannula and a trocar,
wherein the cannula comprises a cannula shaft with a heart-side inlet which comprises an inlet edge which delimits the inlet opening, and with a pump-side outlet and a lumen which extends between the inlet and the outlet, wherein a suture ring for connecting the cannula to the heart is arranged on an outer side of the cannula shaft and the outlet is connectable to a pump; and
wherein the trocar comprises an opening for a guide wire and a trocar shaft which has a puncturing tip and is insertable into the lumen from the outlet, and the trocar shaft is longer than the lumen that in a puncturing configuration the trocar shaft or the puncturing tip completely covers the inlet opening.

17. A method for cannulation of the left-side heart, comprising:
puncturing an apex of a heart with a guide wire;
threading a cannula system comprising a cannula and a trocar, onto the guide wire;
introducing the cannula system up to the apex and puncturing the apex by way of a puncturing tip of the trocar; and
displacing the myocardium by way of the puncturing tip of the trocar and inserting the inlet of the cannula into the ventricle.

18. The method of claim 17 further comprising suturing a suture ring of the cannula to the outer wall of the heart.
